(19) United States
(12) Patent Application Publication
(10) Pub. No.: US 2017/0340153 A1
(43) Pub. Date: Nov. 30, 2017

(54) STRAW DISPENSER WITH IMPROVED STERILIZATION DEVICE

(71) Applicant: STERI-STRAW INC., Sarasota, FL (US)

(72) Inventor: Che Wise-Jarvis, Sarasota, FL (US)

(21) Appl. No.: 15/609,801

(22) Filed: May 31, 2017

Related U.S. Application Data
(60) Provisional application No. 62/352,157, filed on Jun. 20, 2016.
(60) Provisional application No. 62/343,358, filed on May 31, 2016.

(51) Int. Cl. A47G 19/30; B65D 83/02; A61L 2/10

(52) U.S. Cl. CPC A47G 21/184; A61L 2/10; B65D 83/02

(57) ABSTRACT

A straw dispenser having a UV light source for sterilizing individual straws. The straws are metered through a passage. The UV light source is mounted over the passage to sterilize individual straws before delivery to the user. The dispenser may be equipped with an LCD display and communications module.

### CROSS-REFERENCE TO RELATED APPLICATIONS

This application claims priority of U.S. Provisional Application 62/343,358 filed May 31, 2016, and U.S. Provisional Application 62/352,157 filed Jun. 20, 2016, both of which are incorporated herein by reference.

### FIELD OF THE INVENTION

This application relates to straw dispensers and more particularly to straw dispensers with sterilization devices.

### BACKGROUND OF THE INVENTION

Straw dispensers with sterilization devices are known. These devices typically have a housing defining a bin for storing a reservoir of straws. A short wave ultraviolet (UV-C) light source to kill or inactivate microorganisms is located over the straws in the bin. Manual dispensers deliver straws from the bottom of the bin to the user. Automated devices use a continuous belt with outwardly projecting arms to gather straws from the bottom of the bin on the belt. However, the UV light source is unable to irradiate all of the straws in the bin, particularly the straws at the bottom of the bin which are blocked from the UV radiation by straws laying above them. Thus, many of the straws are not properly sterilized.

### SUMMARY OF THE INVENTION

A device for dispensing drinking straws includes a housing having a compartment for containing a plurality of straws, a passage for conveying straws from the compartment to an area outside the housing, at least one partition member extending into the passage to meter one straw at a time from the plurality of straws, and a UV light source disposed directly over the passage to sterilize the one straw as the straw travels through the passage. The device may also include a controller for selectively energizing the UV source when the straw is in the passage beneath the UV source. The device may also include a wireless communications module for sending information regarding usage of the device to a remote location.

### BRIEF DESCRIPTION OF THE DRAWINGS

FIG. 1 is a perspective view of an automated dispenser according to a first preferred embodiment;

FIG. 2 is a cross sectional view of the automated dispenser of the first embodiment;

FIG. 3 is a perspective view of a manual dispenser according to a second preferred embodiment;

FIG. 4 is a partial perspective in view of an interior of the second preferred embodiment with a ramp in the up position;

FIG. 5 is a partial perspective view of an interior of the second preferred embodiment with the ramp in a down position; and

FIG. 6 is a rear perspective view of the interior of the second preferred embodiment.

### DETAILED DESCRIPTION OF THE INVENTION

A device for dispensing straws or other cylindrical objects includes a housing with a compartment holding the objects is disclosed. The objects are metered to pass under a UV source before exiting the housing. In this way each individual straw or object is fully irradiated and sterilized by the UV source one at a time before exiting the housing. The device also includes a communications module for communicating with a remote device to provide information about usage and inventory. Additionally, the device may include an electronic graphic display on the front of the housing.

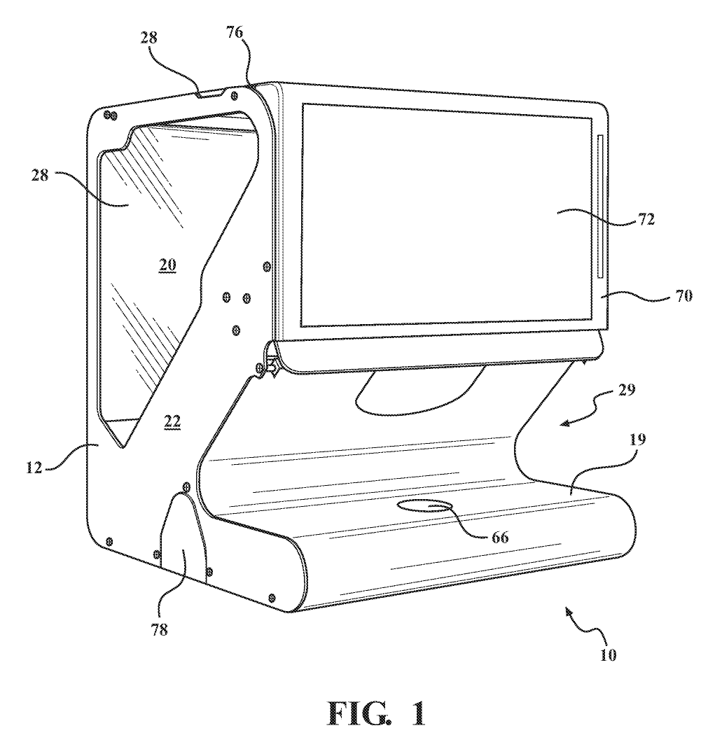

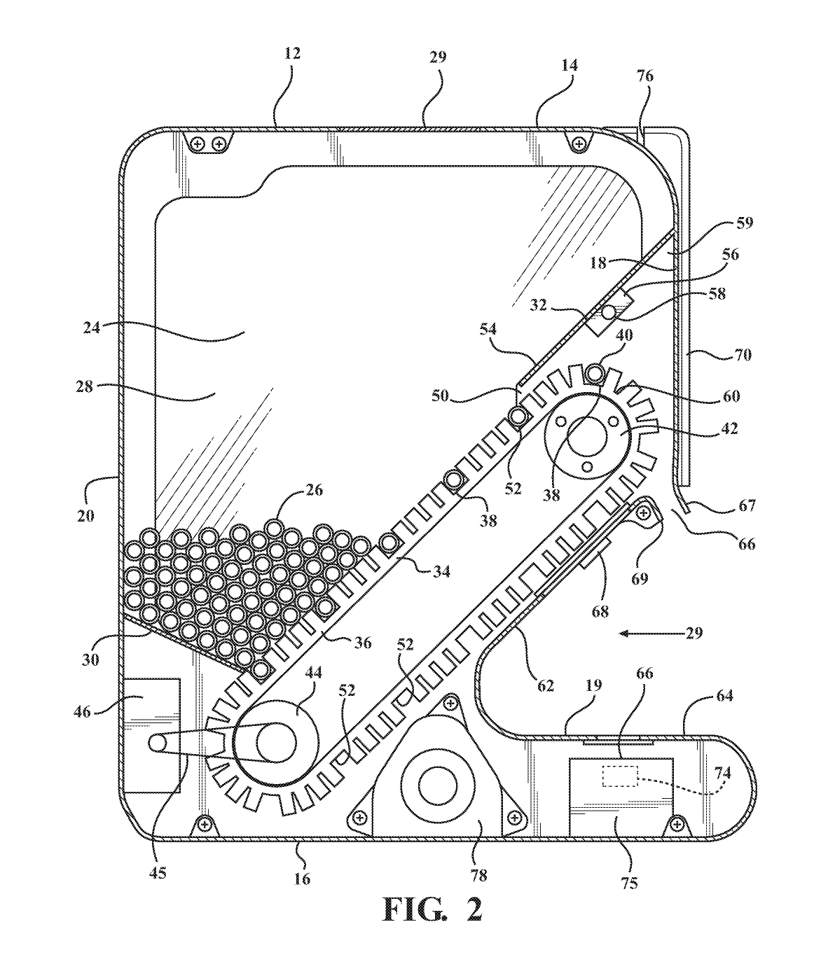

As shown in FIGS. 1 and 2, an automated dispensing device 10 includes a housing 12 having a top 14, bottom 16, upper front wall 18, lower front wall 19, back wall 20, and pair of sidewalls 22. A bin 24 for holding a supply of conventional drinking straws 26 formed between the upper front wall 18, back wall 20 and the pair of sidewalls 22. The top 14 has a removable cover 29 to permit access to the bin 24 for filling of the straws into bin 24. The cover 29 may be provided with a seal such as an o-ring to prevent spilt beverage from entering the bin. The sidewalls 22 have a clear window portion 28 formed of translucent glass or plastic to permit viewing of the straws 26 within the housing 12. The housing 12 is formed of a rigid material such as metal or plastic. The drinking straws 26 are formed of a material which may be fully irradiated by UV light such as a UV transmissive plastic. Each straw has a predetermined outer diameter.

As shown in FIG. 2, the straws 26 are supported within the bin by an angled lower partition 30, an angled upper partition 32, and a conveying member on continuous belt 34. The continuous belt 34 extends upwardly at an angle of approximately 45 degrees from the back wall 20 of the housing between the sidewalls 22 towards the upper front wall 18. The belt has groups 36 of four arms which extend transversely to the direction of travel of the belt. A gap 38 is formed between each group 36 having a width slightly greater than the diameter of the straw. Likewise, the height of each arm of the group 36 is slightly less than the diameter of the straw.

The belt 34 is supported by an idler roller 42 and a drive roller 44. An electric servomotor 46 is connected to the drive roller 44 by a drive belt 45 to propel the belt. Alternatively, the drive motor can be incorporated into the roller. When the belt 34 travels, a single straw 40 falls into the gap 38 between the groups of arms 36 to be carried up the belt into an exit passage 50. The entry of the exit passage 50 is formed between a surface 52 of the belt 34 and the gap 38 formed an end 54 of the upper partition 32. The distance between the partition 32 and surface of the belt is greater than the outer diameter of the straw 40 but less than twice the outer diameter of the straw 40 to meter the straws into the passage so only one straw can pass through the passage.

Angled upper partition 32 and the upper front wall 18 form a compartment 59 for a ultraviolet light source including a fixture 56 holding an ultraviolet lamp 58. The lamp 58 is mounted so as to direct the UV light transversely over a top 60 of the run of the belt. The UV lamp extends the width of the belt 34. Thus, the straws are carried under the end 54 of the upper partition then under the UV light to be fully irradiated and sterilized by the UV light. The straw is carried on the belt 34 over the idler roller 42 until the belt 34 turns downwardly.

The straw 40 drops out of the belt down into a delivery passage 66 formed between a lower end 67 of the upper front wall 18 and an upper end 69 lower front wall 19. The straw 40 exits the delivery passage 66 and drops to a receiving area 29 which is formed by a V shaped portion lower front wall 19. The V shaped portion has an upper portion 62 extending beneath the belt and a lower horizontal portion 64. The receiving area 29 is provided to permit insertion of the user's hand to receive the straw 40. A presence sensor such as an infrared sensor 66 is mounted in the horizontal portion 64. The infrared sensor 66 senses the presence of a hand under the delivery passage 66. Upon sensing the presence of a hand, a signal is sent from the sensor module 66 to a controller 75 which energizes the motor 46 to drive the belt a predetermined distance to dump the straw 40 into the passage 66 and bring a new straw to a position under the light. The controller 75 energizes the light for a predetermined time such as 3 seconds to fully sterilize the straw. The motor 46 drives the belt a predetermined distance equal to a group 36 of four arms and the gap 38. This moves the straw a sufficient distance to travel under the UV light source and drop the straw into the delivery passage 66. A light module 68 which emits blue light such as a blue LED may also be mounted to the housing. The module is mounted above the passage 66 to direct blue light into the receiving area 29 to indicate that the straw has been sterilized.

As shown in FIGS. 1 and 2, the front of the housing 12 may be provided with a display module 70 having an LCD screen 72. The LCD screen 72 displays the vendor's or manufacturer's logo, however can be customized to provide messages, advertising content, or the like. Associated with the screen 72 is a communications module 74 which is mounted within the housing. The communications module 70 receives and delivers information by wireless Internet protocol with a cloud based central control unit. The communications module 74 may also connect by Bluetooth or other short range communication devices to the Internet of Things to control or be controlled with nearby smart apparatus such as HVAC controls, light switches, and the like.

The content to be displayed by the LCD screen 72 may be delivered by an Internet protocol such as Windows 10 and ARM to the communications module 74 and the communications module may be accessed by a mobile device or other Internet connected device. Thus, information, advertising, photographs, and the like can be uploaded from a mobile device or terminal through the communications module 74 and then delivered to a network or to individual dispensers depending upon the desired application. At the same time instructions can be delivered to the dispenser by a mobile app to control nearby smart devices such as the HVAC system, lighting, or the like through the communications module 74. Thus, a store operator through a mobile device and communications module 74 communicate with the dispenser. The communications module then in turn may send signals by Bluetooth or other manner peripheral devices to turn on and off the lights or to change thermostat settings.

A card reader 76 may also be mounted to module 70. The card reader 76 reads credit cards, debit cards, rewards cards, and the like. The card reader allows the dispenser 10 to be a point of sale device and/or to control the dispensing of straws. Information from the card reader is delivered through the communications module 74 to a remote processing system. This allows sales of beverage or other goods to be processed through the dispenser. Likewise, each dispensing operation may be controlled or limited by the machine so that, for instance, only one straw is delivered per sale.

Additionally, the information about usage of the dispenser 10 may be gathered from the controller 75 and delivered by the communications device to a remote location. Information for use in analytics such as the frequency of use and time of use may be gathered. Additionally, the data can be used for keeping track of the inventory of straws in the bin of the dispenser, at an appropriate time, a stock reorder can be sent automatically from the controller or a notification can be sent to the machine's owner to restock the bin with inventory. A near field communications tag (not shown) can be placed on the reader allowing a customer to use the phone to access an offsite location such as the main controller to obtain coupons and other information about the location or menu items offered by the merchant. The servomotor 46, UV light 58, blue light 68, sensor 66 and display module 70 may be powered by a battery 78 or other source of electrical power.

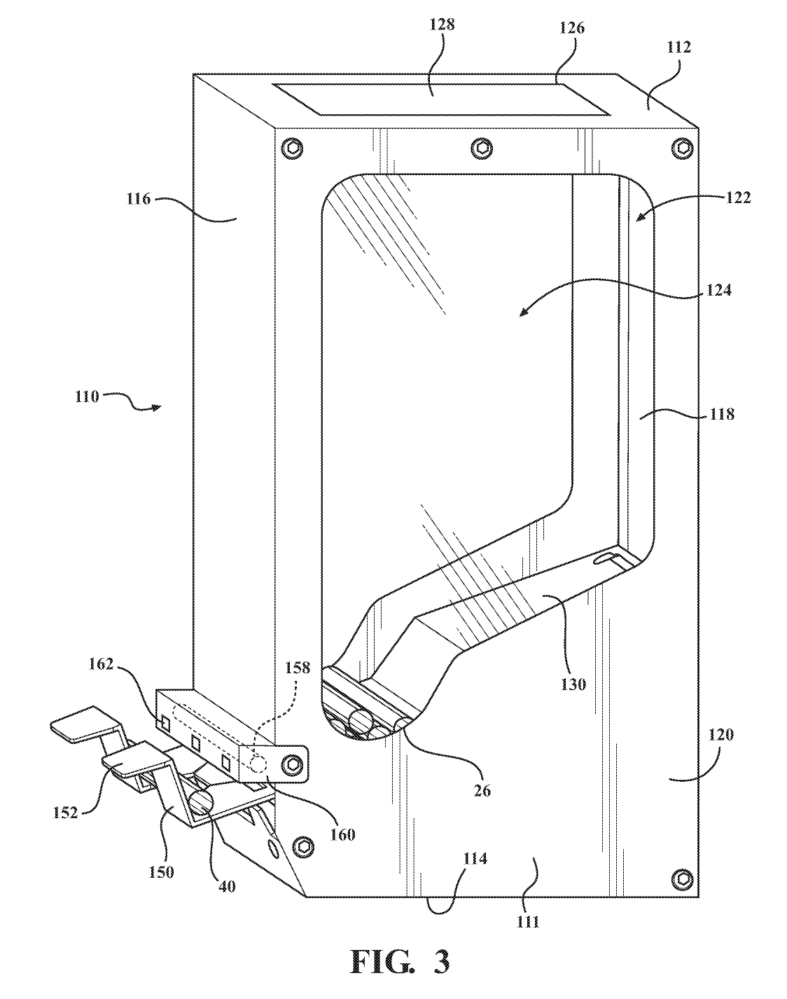

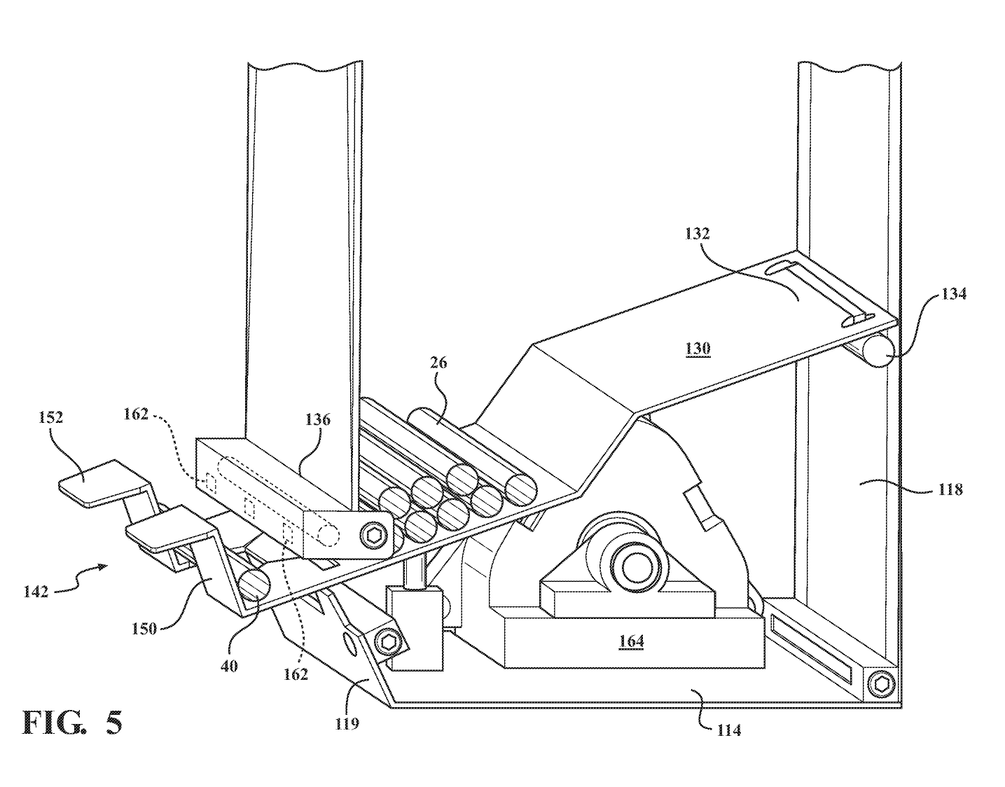

As shown in FIGS. 3-6, a second embodiment of the invention is a manual dispenser 110. The dispenser 110 includes a rectangular housing 111 with a top 112, bottom 114, upper front wall 116, back wall 118, lower wall 119 and a pair of sidewalls 120. Each sidewall 120 has an opening with a pane 122 of glass or plastic. The top 112 has an opening 126 and cover 128 for accessing the bin 124. The width of the bin is slightly greater than the width of the straws so that the straws are aligned with the transverse axis of the housing. A conveying member or ramp 130 angles downwardly across the inside of the housing from the rear wall 118 towards the front wall to define the bottom of the bin 124.

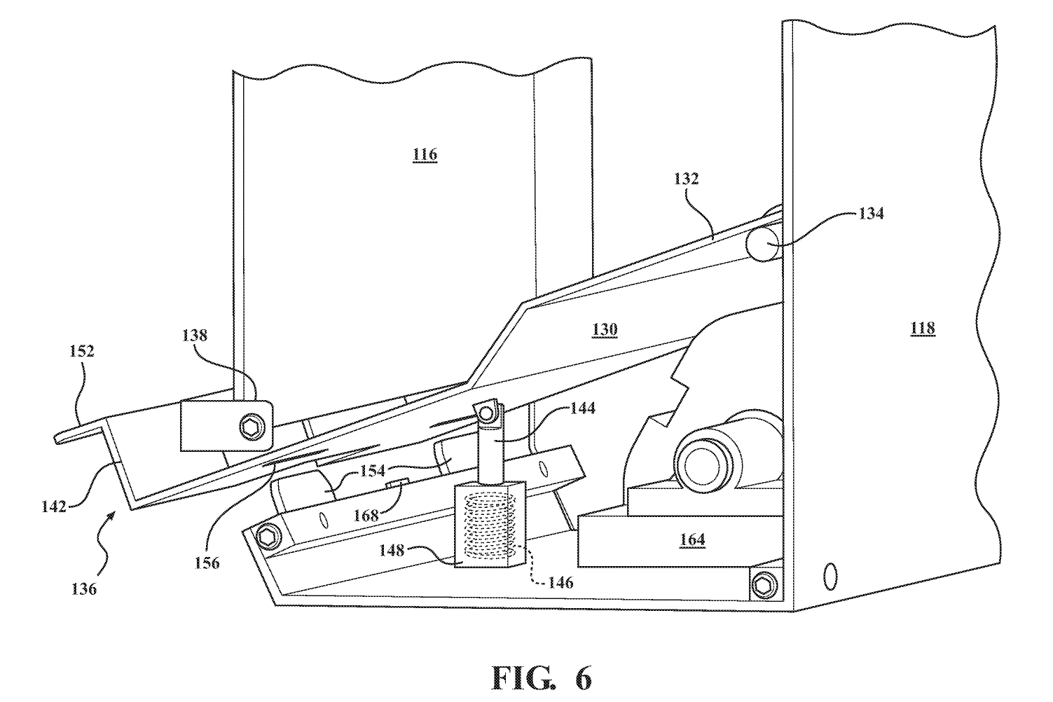

As shown in FIG. 6, the ramp 130 is biased upwardly against a partition formed by the lower end 136 of the upper front wall 116 on arm 144. The arm 144 is biased upwardly by springs 146 held in a spring holder 148. The partition holds the straws from rolling down the ramp 130 to exit the bin 124. A lower end 142 of the ramp 130 has an upwardly extending arms 150 and a flange 152 for depressing the ramp. The arms 150 catch the straws. A pair of flanges 154 extend upwardly from a lower wall 119 to hold straws in the bin when the ramp 130 is depressed. The flanges 154 pass through slots 156 in the ramp 130 the front wall 116 when the ramp is depressed. The flanges 154 are spaced inwardly from the front walls a distance generally equal to a width of a straw. When the ramp is depressed, the flanges 154 rise through the slots 156 the separate a straw 40 which rests against the partition from the remaining straws in the bin. Once the ramp is depressed a distance greater than the width of a straw 40 at the partition, the straw rolls down the ramp under a UV light source mounted in a fixture 160 to the outside of the front wall of the housing over the ramp. The fixture can be just inside the housing or beneath the wall 116.

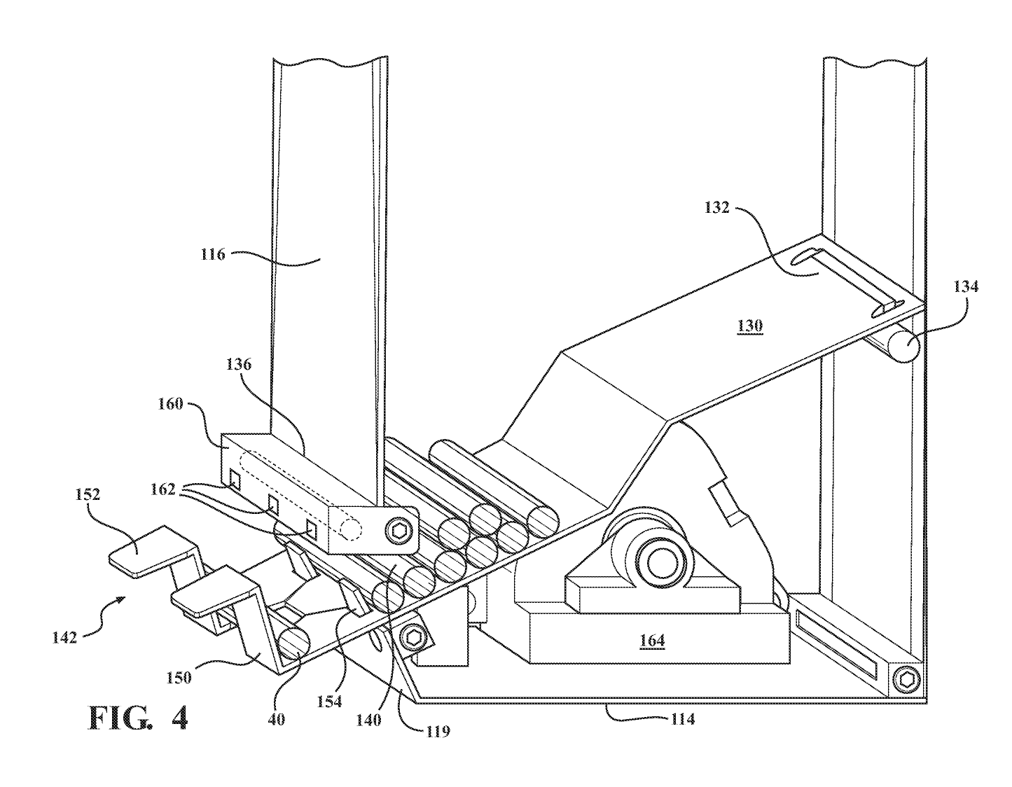

As shown in FIGS. 3 and 4, the fixture 160 holds the ultraviolet lamp 158. The fixture is mounted to the upper front wall 116 to direct the light from the lamp 158 downwardly to irradiate the straw 40 as it passes down an exit passage from the bin to the receiving area formed by upwardly extending portion 150 of the ramp 130. A blue light source such as an LED 162 may be mounted in the fixture 160 to direct light onto the receiving area. The light will indicate that the straw is sterilized and that the receiving area is safe for grabbing the straw. A power module such as a rechargeable battery pack 164 or a cord for electrical connection is mounted in the housing to power the light.

As shown in FIG. 6, a switch 168 is mounted beneath the ramp 130. When the ramp 130 is depressed the switch 168 is closed to provide electrical power from the power supply to energize the ultraviolet light 158 to sterilize the straw 40 as it passes down the passage.

The dispenser may be provided with the display module 70 which is disclosed above for the automated dispenser above. The power supply also supplies power for the controller and screen.

Thus is disclosed two embodiments of the invention by which straws are sterilized fully and completely just before they pass into the user's hands. It will be apparent to those skilled in the art that many variations of the invention are within the scope of the disclosure.

1. A device for dispensing drinking straws, comprising:
a housing having a bin for containing a plurality of straws;
at least one partition member extending towards the conveying member to form an exit possible to meter one straw from the plurality of straws in the bin.
a conveying member for conveying straws from the bin to an area outside of the housing;
a UV source disposed over conveying member to sterilize the one straw as the one straw travels from the bin to the outside of the housing.

2. The device of claim 1 further comprising:
a controller for selectively energizing the UV source when the one straw is in the passage beneath the UV source.

3. The device of claim 1 comprising a wireless communications for sending and or receiving information regarding usage to a remote location.

4. The device of claim 1 wherein the passage has a surface and an upper partition spaced apart a predetermined distance to define an entry into the passage.

5. The device of claim 4 wherein each straw of the plurality of straws has a predetermined diameter and the predetermined distance defining the opening is greater than the predetermined diameter of the straw and less than twice the predetermined diameter of the straw.

6. The device of claim 4 wherein the lower surface is provided by a conveyor belt.

7. The device of claim 6 wherein the at least one partition extends from the conveyor belt.

8. The device of claim 4 wherein the lower surface is provided on an inclined member.

9. The device of claim 8 wherein the inclined member has at least one slot and the at least one partition member passes through the slot.

10. The device of claim 1 further comprising:
an electronic display mounted to the housing for displaying information.
